(19) United States
(12) Patent Application Publication
(10) Pub. No.: US 2012/0310283 A1
(43) Pub. Date: Dec. 6, 2012

(54) SEGMENTAL SPINAL FIXATION SYSTEM AND A METHOD OF FIXATING A PLURALITY OF SPINAL SEGMENTS

(76) Inventor: Vittorio M. Morreale, Rochester, MI (US)

(21) Appl. No.: 13/481,033

(22) Filed: May 25, 2012

Related U.S. Application Data
(60) Provisional application No. 61/492,483, filed on Jun. 2, 2011.

(51) Int. Cl. A61B 17/70; A61B 17/88

(52) U.S. Cl. 606/256; 606/279

(57) ABSTRACT

A spinal fixation system includes a plurality of segmental members each having a concave end and a convex end, and aligned end to end with the convex end of one of the segmental members disposed within the concave end of an adjacent segmental member to define a segmental rod. The rod is formed for attachment to a plurality of spinal segments, and then tensioned to generate a stiffness in the segmental rod sufficient to maintain the pre-formed shape. A locking mechanism secures a tensile connector to the segmental rod and maintains the tension of the tensile connector once secured and the shape of the preformed segmental rod. The locking mechanism includes a crimping device through which the tensile connector passes, and a guide device through which the tensile connector wraps around. The crimping device is compressed to grasp and secure the tensile connector in place relative to the segmental rod.

### CROSS-REFERENCE TO RELATED APPLICATIONS

This application claims the benefit of U.S. Provisional Patent Application Ser. No. 61/492,483, filed on Jun. 2, 2011, the disclosure of which is hereby incorporated by reference.

### TECHNICAL FIELD

The invention generally relates to a segmental spinal fixation system for securing a position of a plurality of spinal segments, i.e., vertebrae, and to a method of positioning the plurality of spinal segments with the spinal fixation system.

### BACKGROUND

Human spinal segments, i.e., vertebrae, and their associated connective elements are subject to a variety of diseases, conditions and/or injuries that cause pain and/or disability, including but not limited to degenerative spine disease, unstable spine, scoliosis, cancer and other diseases, or vertebrae fractures. A variety of systems and methods have been developed for treating these conditions. For example, spinal fixation systems are used to stabilize spinal segments in need of stabilization, and to promote bone growth between the spinal segments to fuse the spinal segments together.

Conventional spinal fixation systems include a plurality of support/attachment assemblies that support and attach a rigid rod to a plurality of spinal segments. The rod requires bending to conform to the spinal segments and the support/attachment assemblies. The rod is very rigid and requires a significant force to bend into the proper shape. In order to properly fit the rod to the spinal segments, the rod is bent into two planes, the sagittal plane and the frontal plane. Bending of the rod in both the sagittal plane and the frontal plane is a complex and time consuming operation. At times, the rod must be bent after the rod is attached to the support/attachment assemblies and the spinal segments. The bending of the rod while attached to a patient during a surgical intervention is difficult and poses potential risks to the patient due to the force required to bend the rod and the possibility of bending tools slipping and impacting the vertebral column, the spinal cord and/or nerves. The potential risks to the patient include potential spinal cord and nerve injuries.

Segmental rods may be substituted for the rigid rods described above. The segmental rods include multiple members, each having a concave end and a convex end, that are laid end to end with the convex end of one segmental member disposed within the concave end of an adjacent segmental member such that the segmental rod may be more easily manipulated into the proper position. The segmental members of the segmental rod are held together by a tensile connector, such as a cable or the like, that runs through a center of each of the segmental members.

### SUMMARY

A segmental spinal fixation system is provided. The segmental spinal fixation system includes at least a first segmental member and a second segmental member. Each of the first segmental member and the second segmental member include a convex end and a concave end. Each of the first segmental member and the second segmental member define a central bore that extends longitudinally. The first segmental member and the second segmental member are disposed adjacent each other to define a segmental rod extending along a longitudinal axis. The first segmental member and the second segmental member are aligned such that the convex end of one of the first segmental member and the second segmental member is disposed within the concave end of the other of the first segmental member and the second segmental member. The segmental spinal fixation system further includes a tensile connector that extends through the central bore of each of the first segmental member and the second segmental member. A locking mechanism is disposed at an axial end of the segmental rod. The locking mechanism includes a crimping device and a guide device. The crimping device defines a passage, and is disposed adjacent the axial end of the segmental rod. The guide device is disposed adjacent the crimping device. The tensile connector extends through the passage of the crimping device, around an outer periphery of the guide device, and extends back through the passage of the crimping device thereby forming a loop around the guide device. The crimping device is disposed in frictional engagement with the tensile connector to secure the tensile connector relative to the segmental rod.

A method of positioning a plurality of spinal segments with a spinal fixation system is also provided. The method includes attaching a tensile connector to a first axial end of a segmental rod having a plurality of segmental members. The tensile connector is passed through a central bore in each of the plurality of segmental members to position the plurality of segmental members relative to each other and define the segmental rod. The segmental rod is fixed to each of the plurality of spinal segments. The method further includes aligning the plurality of spinal segments into a desired position, and tensioning the tensile connector. The tensile connector is tensioned to a tension force sufficient in magnitude to generate a pre-determined stiffness in the segmental rod. A locking mechanism is engaged to secure the tensile connector relative to a second axial end of the segmental rod and to maintain the tension within the tensile connector. Engaging the locking mechanism includes passing an end of the tensile connector through a crimping device, wrapping the tensile connector around a guide device, passing the end of the tensile connector back through the passage in the crimping device to thereby form a loop around the guide device, and compressing the crimping device over the tensile connector. The crimping device is compressed over the tensile connector to secure the position of the tensile connector relative to the segmental rod while maintaining the tension in the tensile connector.

Accordingly, the locking mechanism for the spinal fixation system provides a quick and convenient manner in which to secure the tensile connector relative to the segmental rod while maintaining the tension within the tensile connector.

The above features and advantages and other features and advantages of the present invention are readily apparent from the following detailed description of the best modes for carrying out the invention when taken in connection with the accompanying drawings.

### BRIEF DESCRIPTION OF THE DRAWINGS

FIG. 1 is a schematic perspective view of a spinal fixation system attached to a plurality of spinal segments.

FIG. 2 is a schematic cross sectional view of the spinal fixation system.

FIG. 2A is a schematic cross sectional view of an alternative embodiment of the spinal fixation system.

FIG. 2B is a schematic cross sectional view of a second axial end of the spinal fixations system shown in FIG. 2A.

FIG. 3 is a schematic cross sectional view of a single segmental member of the spinal fixation system.

FIG. 4 is a schematic perspective view of the single segmental member of the spinal fixation system.

FIG. 5 is a schematic plan view of a locking mechanism of the spinal fixation system.

FIG. 6 is an exploded schematic plan view of the locking mechanism of the spinal fixation system.

FIG. 7 is a schematic perspective view of a crimping device of the locking mechanism.

FIG. 8 is a schematic plan view of the crimping device of the locking mechanism.

FIG. 9 is a schematic plan view of a guide device of the locking mechanism.

FIG. 10 is a schematic plan view of the guide device of the locking mechanism.

FIG. 11 is a schematic cross sectional view of the guide device of the locking mechanism.

FIG. 12 is a schematic plan view of an alternative embodiment of one of the segmental members of the spinal fixation system.

FIG. 13 is a schematic perspective view of an end segmental member in which a crimping device of the locking mechanism is integrally formed with a segmental member of the spinal fixation device.

FIG. 14 is a schematic plan view of the end segmental member shown in FIG. 13.

FIG. 15 is a schematic plan view of an alternative embodiment of the spinal fixation system showing a plurality of tension zones, each having a zone tensioning segmental member and a locking mechanism for tensioning a tensile connector to a different tension in each of the different tension zones.

FIG. 16 is a schematic cross sectional view of a lockable segmental member of the alternative embodiment of the spinal fixation system shown in FIG. 15.

FIG. 17 is a schematic cross sectional view of a zone tensioning segmental member of the alternative embodiment of the spinal fixation system shown in FIG. 15.

### DETAILED DESCRIPTION

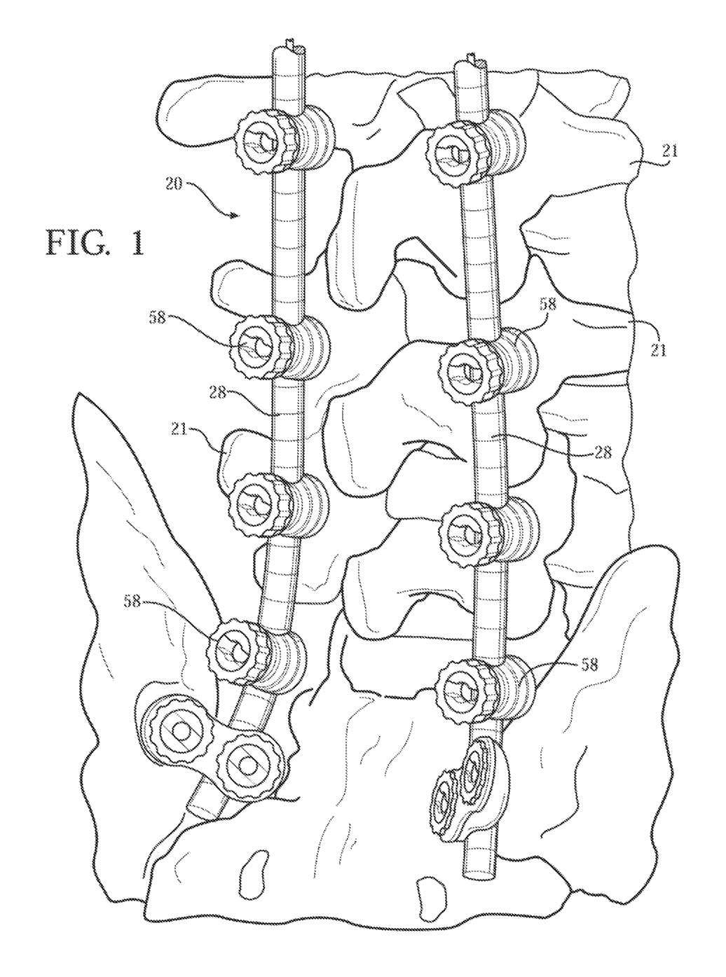

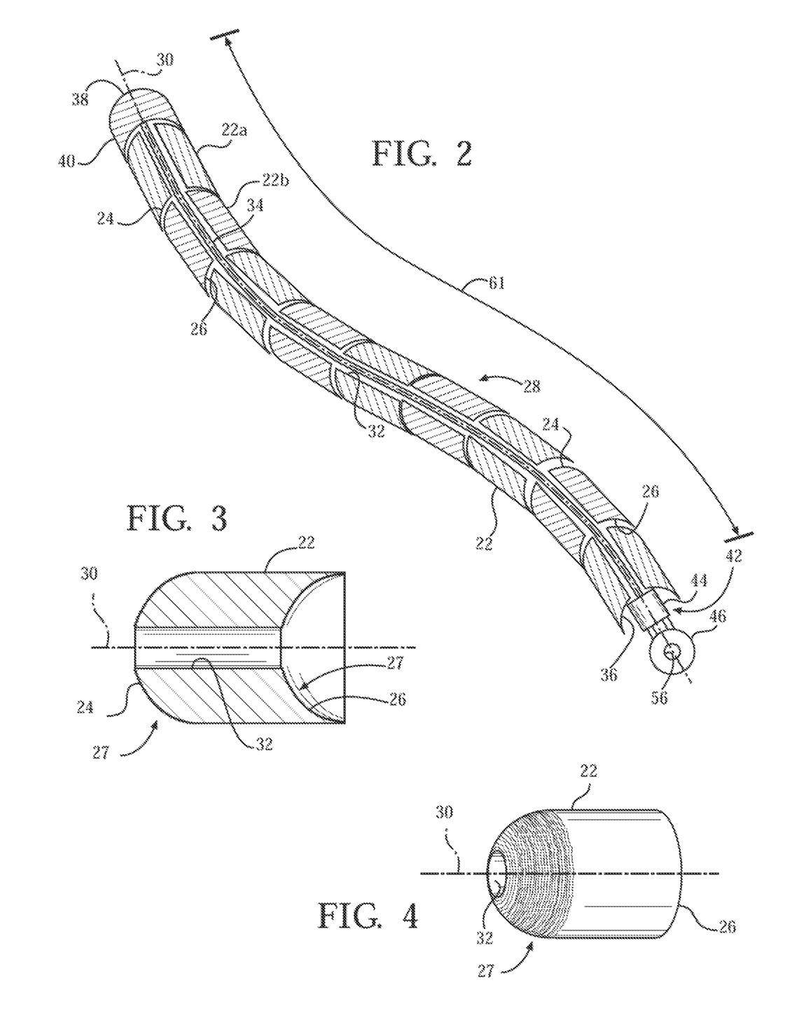

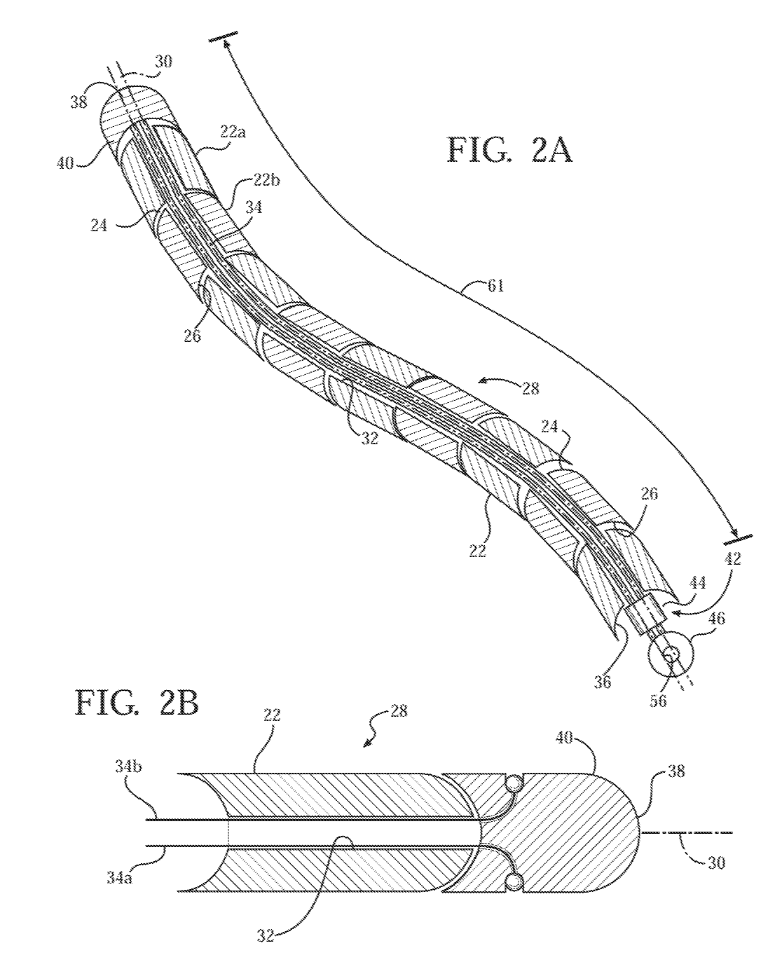

Referring to the Figures, wherein like numerals indicate like parts throughout the several views, a segmental spinal fixation system is generally shown at 20 in FIG. 1. Referring to FIGS. 1 and 2, the spinal fixation system 20 couples a plurality of spinal segments 21, i.e., vertebrae, together.

The spinal fixation system 20 includes a plurality of segmental members 22. The plurality of segmental members 22 includes at least a first segmental member 22a and a second segmental member 22b. Referring also to FIGS. 3 and 4, each of the segmental members 22 includes a convex end 24 and a concave end 26. The plurality of segmental members 22 are disposed end to end to define a segmental rod 28 that extends along a longitudinal axis 30. As shown, the first segmental member 22a and the second segmental member 22b are disposed adjacent each other, and are aligned such that the convex end 24 of one of the first segmental member 22a and the second segmental member 22b is disposed within the concave end 26 of the other of the first segmental member 22a and the second segmental member 22b.

The concave end 26 and the convex end 24 of the segmental members 22 each include a semi-spherical shape that is designed to mate with each other and provide a pivotal interface therebetween. The pivotal interface allows the segmental rod 28 to be positioned, e.g., bent, in both the sagittal plane and the frontal plane. Additionally, as best shown in FIGS. 3 and 4, one or each of the concave end 26 and the convex end 24 of the segmental members 22 may include a textured surface 27 for engaging each other in frictional engagement to prevent relative movement between the segmental members 22 once positioned in place and secured together as described in greater detail below. The frictional surface 27 may include annular ridges, dimples, a roughed surface, or some other surface irregularity capable of increasing friction.

Referring to FIGS. 2 and 3, each of the segmental members 22 defines a central bore 32 extending longitudinally therethrough. Preferably, each of the segmental members 22 includes a generally cylindrical body, with the central bore 32 concentric with a central axis of the cylindrical body. However, it should be appreciated that the body of the segmental members 22 need not be cylindrical in shape.

Referring to FIG. 2, a tensile connector 34 extends through the central bore 32 of each of the segmental members 22. The tensile connector 34 may include but is not limited to one of a cable or a wire. FIG. 2 shows a single tensile connector 34 extending through the central bore 32 of the segmental members 22. However, referring to FIG. 2A, the segmental rod 28 is shown with two tensile connectors 34 extending through the central bore 32 of the segmental members 22. Accordingly, it should be appreciated that the tensile connector 34 may include multiple tensile connectors 34. The tensile connector 34 is fixedly attached to an axial end of the segmental rod 28. As shown, the segmental rod 28 includes a first axial end 36 and a second axial, with the tensile connector 34 fixedly attached to the second axial end 38 of the segmental rod 28. The tensile connector 34 may be attached to the segmental rod 28 at the second axial end 38 in any suitable manner. For example, the tensile connector 34 may include a cap 40 disposed at an end of the tensile connector 34 that includes a diameter greater in size than the central bore 32 of the segmental members 22 to prevent the tensile connector 34 from being pulled through the central bore 32 of the segmental members 22.

Referring to FIG. 2B, the second axial end 38 of the segmental rod 28 is shown with a cap 40 attached to a first tensile connector 34a and a second tensile connector 34b. The ends of the first tensile connector 34a and the second tensile connector 34b are attached to the cap 40 along a common plane and positioned 180° apart from each other. The ends of the first tensile connector 34a and the second tensile connector 34b are attached to the cap 40 near an outer peripheral surface of the cap so as to be spaced as far apart from each other as possible. The opposite ends of the first tensile connector 34a and the second tensile connector 34b respectively are secured by a locking mechanism (such as shown in FIG. 2A). The locking mechanism 42 may apply tension to either one of the first tensile connector 34a or the second tensile connector 34b to move the cap 40 clockwise or counterclockwise along the common plane as viewed on the page of FIG. 2B, i.e., to steer the cap 40. Accordingly, by tensioning one of the first tensile connector 34a or the second tensile connector 34b, the shape of the segmental rod 28 may be changed while within the body to steer the segmental rod 28 into position and properly align the segmental rod 28 along the spinal segments 21. When the segmental rod 28 is positioned in a final position, then the locking mechanism 42 may be tensioned equally against both the first tensile connector 34a and the second tensile connector 34b to secure the segmental rod 28 in position. It should be appreciated that three dimensional steering of the cap 40 may be achieved by using three or more tensile connectors 34, properly positioned around a central axis of the cap 40.

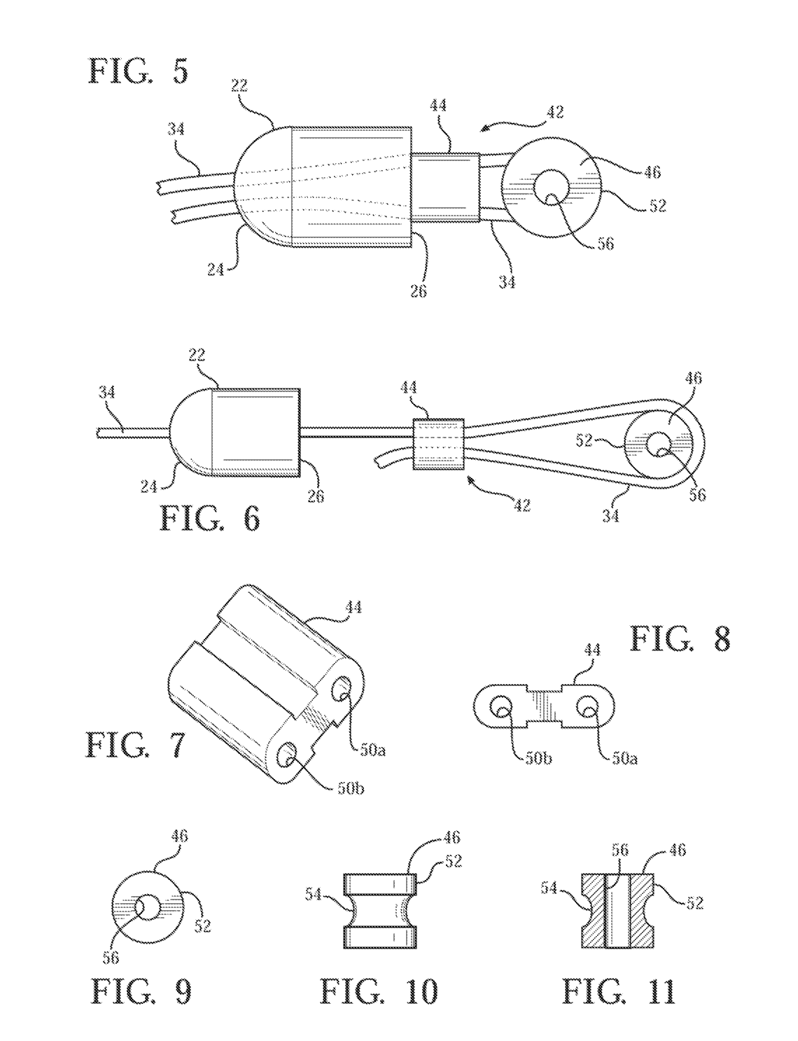

Referring to FIGS. 5 and 6, a first locking mechanism 42 is disposed at an axial end of the segmental rod 28. The first locking mechanism 42 secures the tensile connector 34 relative to the segmental rod 28. As shown, the tensile connector 34 is disposed at the first axial end 36 of the segmental rod 28. The first locking mechanism 42 includes a crimping device 44 and a guide device 46. As shown in FIG. 5, the crimping device 44 and the tensile connector 34 are configured to provide a double stranded tensile connector 34, such as shown in FIG. 2A. As shown in FIG. 6, the crimping device 44 and the tensile connector 34 are configured to provide a single stranded tensile connector 34, such as shown in FIG. 2.

The crimping device 44 is disposed immediately adjacent to the first axial end 36 of the segmental rod 28. As shown in FIGS. 5 and 6, the crimping device 44 is separate from the segmental rod 28. However, as shown in FIGS. 13 and 14, the crimping device 44 may be integrally formed with one of the segmental members 22 to define an end segmental member 48 that is disposed at one of the first axial end 36 or the second axial end 38 of the segmental rod 28.

Referring to FIGS. 7 and 8, the crimping device 44 defines a passage 50. As shown, the passage 50 of the crimping device 44 includes a first passage 50a and a second passage 50b. The first passage 50a is configured for engaging a first portion of the tensile connector 34, and the second passage 50b is configured for engaging a second portion of the tensile connector 34. However, it should be appreciated that the passage 50 may include only a single passage 50. The passage 50, prior to compression thereof, includes a diameter large enough to allow the tensile connector 34 to pass therethrough.

Referring to FIGS. 9 through 11, the guide device 46 is disposed adjacent the crimping device 44. The guide device 46 includes a body having an outer surface 52. The outer surface 52 of the guide device 46 defines a groove 54 that is configured for receiving and positioning the tensile connector 34 therein. Once the first locking mechanism 42 is secured, the groove 54 prevents the tensile connector 34 from moving relative to the body of the guide device 46. The guide device 46 may include a tool engaging mechanism 56. The tool engaging mechanism 56 is configured for engaging a tensioning/crimping tool (not shown). The tensioning/crimping tool is a tool used to tension the tensile connector 34 and/or simultaneously crimp, i.e., compress, the crimping device 44 onto and against the tensile connector 34. As such, the tensioning/crimping tool pulls on the tensile connector 34 to tension the tensile connector 34, while crushing the passage 50 of the crimping device 44 onto and against the tensile connector 34 to secure the tensile connector 34 relative to the crimping device 44. As shown in FIGS. 9 and 11, the tool engagement mechanism includes a threaded aperture extending through the body of the guide device 46. The threaded aperture is oriented perpendicularly relative to the longitudinal axis 30 of the segmental rod 28. As shown in FIGS. 13 and 14, the tool engagement mechanism 56 is disposed on the end segmental member 48, and includes a plurality of detents disposed in the body of the end segmental member 48. It should be appreciated that the tool engagement mechanism 56 may include some other mechanism not shown or described herein that is capable of engaging and/or interacting with the tensioning/crimping tool.

Referring to FIGS. 5 and 6, the tensile connector 34 extends through the central bore 32 of the segmental members 22, and into and through the first passage 50a of the crimping device 44. The tensile connector 34 further extends around the outer periphery of the guide device 46 within the groove 54, and back through the second passage 50b of the crimping device 44. In so doing, the tensile connector 34 forms a loop around the guide device 46. The tensile connector 34 is then brought to the desired tension. After which, the crimping device 44, and thereby the first passage 50a and the second passage 50b, is then compressed to secure the tensile connector 34 within the first passage 50a and the second passage 50b respectively, and secure the tensile connector 34 relative to the crimping device 44. Once compressed, the crimping device 44 is disposed in frictional engagement with the tensile connector 34 to secure the tensile connector 34 relative to the segmental rod 28.

Once tensioned and secured relative to the segmental rod 28, the tensile connector 34 includes a tension force that is sufficient in magnitude to draw the segmental members 22, including the first segmental member 22a and the second segmental member 22b, together. The tension force generates a friction force between the segmental members 22 that is sufficient in magnitude to generate a pre-determined stiffness against bending in the segmental rod 28. Accordingly, the greater the tension in the tensile connector 34, the greater the stiffness of the segmental rod 28. The tensile connector 34 is tensioned to the required tension in order to provide the required stiffness in the segmental rod 28 to resist bending of the segmental rod 28.

As best shown in FIG. 2, the spinal fixation system 20 includes a single tension zone 61 that extends between the first axial end 36 and the second axial end 38 of the segmental rod 28. The single tension zone 61 is created by tensioning the tensile connector 34 with the first locking mechanism 42 disposed at the first axial end 36 of the segmental rod 28, while the opposite end of the tensile connector 34 is fixed to the second axial end 38 of the segmental rod 28.

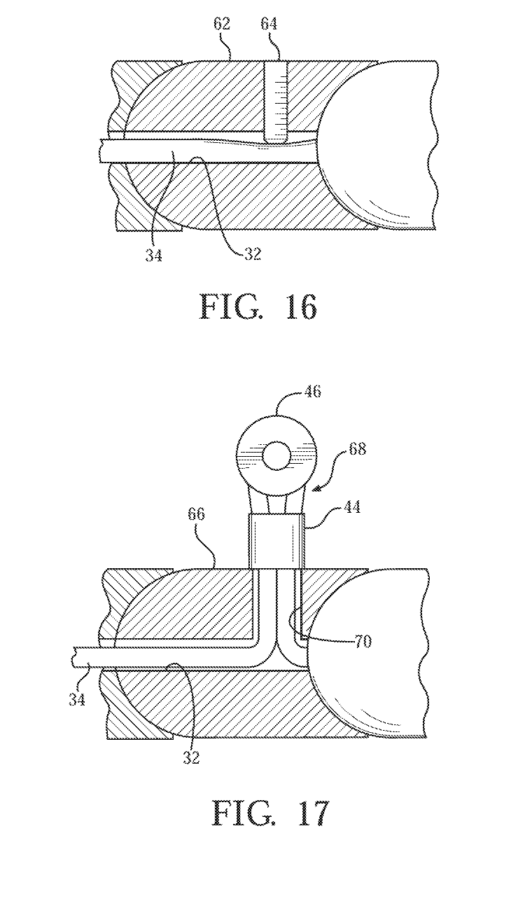

However, referring to FIG. 15, an alternative embodiment of the spinal fixation system is generally shown at 60. The spinal fixation system 60 is configured to provide a plurality of tension zones 61a, 61b, 61c, 61d, 61e. In order to define the plurality of tension zones 61a-61e, a lockable segmental member 62 is positioned within the plurality of segmental members 22. The lockable segmental member 62 is shown in FIG. 16. The lockable segmental member 62 includes a fixing device 64 for securing the tensile connector 34 relative to the lockable segmental member 62. The lockable segmental member 62 is disposed between the first axial end 36 of the segmental rod 28 and the second axial end 38 of the segmental rod 28 to define a plurality of tension zones 61. As shown in FIG. 15, multiple lockable segmental members 22 may be positioned within the plurality of segmental members 22 to define the multiple tension zones 61a-61e. The fixing device 64 may include but is not limited to a set screw in threaded engagement with the body of the lockable segmental member 62. The set screw is configured for compressing the tensile connector 34 within the central bore 32 to secure the tensile connector 34 in place relative to the lockable segmental member 62. By securing the tensile connector 34 relative to the lockable segmental member 62, tension within the tensile connector 34 may not pass beyond the lockable segmental member 62.

In order to provide the required tension within each of the tension zones 61, one of the plurality of segmental members 22 includes a zone tensioning segmental member 66. The zone tensioning segmental member 66 is shown in FIG. 17. Each of the tension zones 61, other than tension zones 61 disposed adjacent an axial end of the segmental rod 28 next to the first locking mechanism 42, includes one zone tensioning segmental member 66. The zone tensioning segmental member 66 allows for a second locking mechanism 68 to engage the tensile connector 34 and secure the tensile connector 34 relative to the zone tensioning segmental member 66 to maintain the tension within the tensile connector 34. The zone tensioning segmental member 66 defines a lateral bore 70 that extends laterally from the central bore 32 through the body of the zone tensioning segmental member 66. The tensile connector 34 may be drawn through the lateral bore 70 so that the second locking mechanism 68 may engage the tensile connector 34. The second locking mechanism 68 is configured and operates in the same manner as the first locking mechanism 42 described above and shown in FIGS. 5 through 11.

The second locking mechanism 68 is disposed adjacent the lateral bore 70 of the zone tensioning segmental member 66. The tensile connector 34 extends through the central bore 32 of the zone tensioning segmental member 66, through the lateral bore 70 of the segmental member 22, through the passage 50 of the crimping device 44 of the second locking mechanism 68, around an outer periphery of the guide device 46 of the second locking mechanism 68, back through the passage 50 of the crimping device 44 of the second locking mechanism 68, back through the lateral bore 70 of the zone tensioning device, and continues through the central bore 32 of the zone tensioning segmental member 66, thereby forming a loop around the guide device 46 of the second locking mechanism 68. Once positioned, the tensile connector 34 is brought to the desired tension to generate the desired stiffness within that specific tension zone 61. Once the tensile connector 34 is properly tensioned, the crimping device 44 of the second locking mechanism 68 is then compressed to crush the passage 50 of the crimping device 44 over the tensile connector 34, thereby securing the tensile connector 34 relative to the crimping device 44. The crimping device 44 is thereby disposed in frictional engagement with the tensile connector 34 to secure the tensile connector 34 relative to the zone tensioning segmental member 66 and maintain the tension within that tension zone 61. As shown in FIG. 15, each tension zone 61 between lockable segmental members 22 includes a zone tensioning segmental member 66 and a second locking mechanism 68 to define and maintain the tension within each of the specific tension zones 61.

The arrangement of the spinal fixation system 60 shown in FIG. 15 allows for the segmental rod 28 to define a different stiffness at different locations along the segmental rod 28. As such, one portion of the segmental rod 28 may be designed to include a tension zone 61 having a lighter stiffness to allow minor flexure along the segmental rod 28, while adjoining sections of the segmental rod 28 may include tension zones 61 designed to include a higher stiffness to prevent any flexure of the segmental rod 28.

Each of the segmental members 22, the tensile connector 34, the crimping device 44 and the guide device 46 are manufactured from a material suitable for disposition within a human body, including but not limited to stainless steel or some other suitable material.

A method of positioning the plurality of spinal segments 21 is also provided. The method includes attaching the tensile connector 34 to an axial end of the segmental rod 28. As described above, the tensile connector 34 may be attached to the axial end in any suitable manner. The manner of attaching the tensile connector 34 to the axial end of the segmental rod 28 need only resist a tensional force in the tensile connector 34.

The method further includes passing the tensile connector 34 through the central bore 32 in each of the plurality of segmental members 22. The tensile connector 34 is passed through each of the aligned segmental members 22 to position the plurality of segmental members 22 relative to each other and define the segmental rod 28.

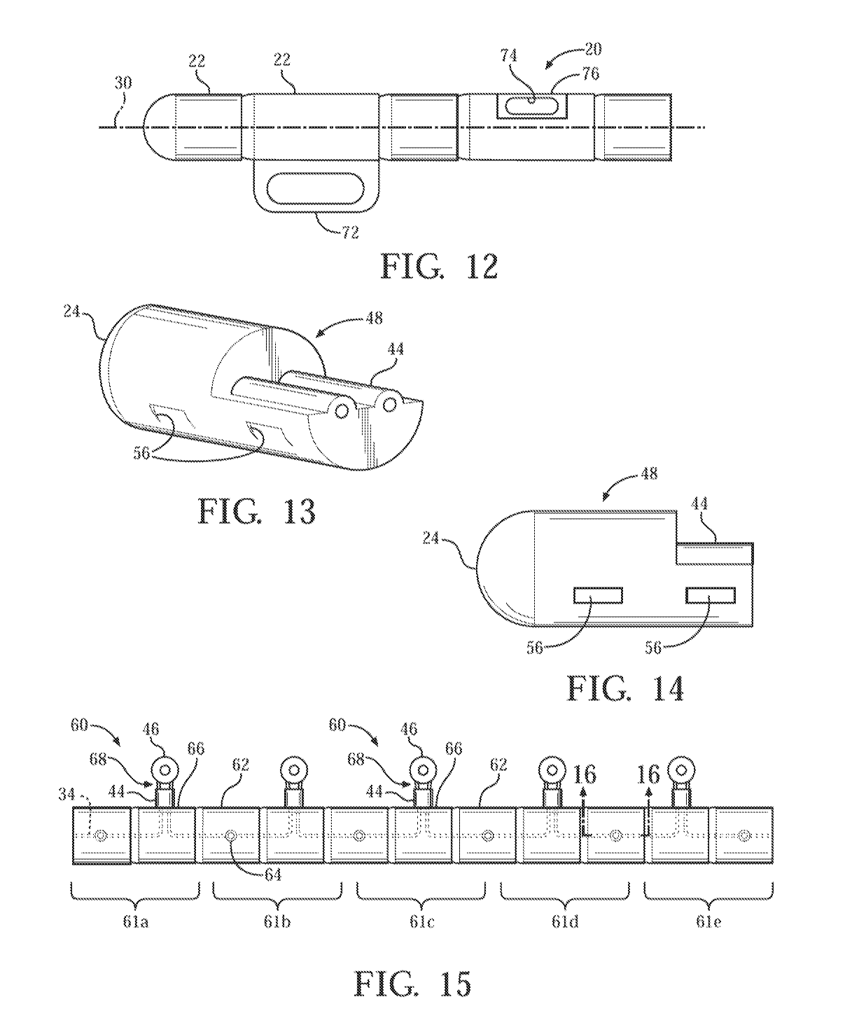

Once the segmental members 22 are positioned relative to each other with the tensile connector 34 passing through the central bore 32 of each of the segmental members 22, the method includes fixing the segmental rod 28 to each of the plurality of spinal segments 21. The segmental rod 28 may be fixed, i.e., attached to the spinal segments 21 in any suitable manner, including but not limited to attaching the segmental rod 28 to the spinal segments 21 with a plurality of attachment assemblies 58, including but not limited to bone screws or some other similar device. The spinal fixation system 20 described herein is suitable for use with many of the attachment assemblies 58 known in the art for attaching spinal support rods to spinal segments 21. In order to accommodate some of the attachment assemblies 58 known in the art, the segmental members 22 may include a mounting bracket 72, or may define an aperture 74 recessed into a flat surface 76 defined by the segmental member 22, such as shown in FIG. 12.

The method further includes aligning the plurality of spinal segments 21 into a desired position. The spinal segments 21 are aligned by manipulating the spinal segments 21 and/or the segmental rod 28 in both the sagittal plane and/or the frontal plane until in the desired orientation. It should be appreciated that the segmental rod 28 may be shaped and pre-tensioned to a desired tension prior to attachment to the spinal segments 21 such that the spinal segments 21 are aligned when attached to the segmental rod 28. Alternatively, the segmental rod 28 may first be attached to the spinal segments 21, after which the segmental rod 28 and spinal segments 21 are manipulated into the desired position.

The method may further include tensioning the tensile connector 34 after the segmental rod 28 and the tensile connector 34 are fixed to the spinal segments 21. The tensile connector 34 is tensioned to a tension sufficient in magnitude to generate a pre-determined stiffness in the segmental rod 28. As noted above, the stiffness in the segmental rod 28 is directly related to the tension in the tensile connector 34. Accordingly, in order to achieve a higher stiffness in the segmental rod 28, the tensile connector 34 is tensioned to a higher tension that corresponds with and achieves the desired stiffness in the segmental rod 28. It should be appreciated that an increase in the tension in the tensile connector 34 increases the rigidity of the spinal fixation system 20, and that a decrease in the tension in the tensile connector 34 decreases the rigidity of the spinal fixations system 20. Accordingly, the tensile connector 34 may be tensioned to provide a very rigid segmental rod 28, e.g., with a high tension, or alternatively, the tensile connector 34 may be tensioned to provide a semi-flexible segmental rod 28, e.g., with a lower tension.

Once the tensile connector 34 is properly tensioned, the method further includes engaging the first locking mechanism 42 to secure the tensile connector 34 relative to an axial end of the segmental rod 28, and to maintain the tension within the tensile connector 34. The first locking mechanism 42 is engaged by a tensioning/crimping tool as described above to both tension the tensile connector 34 and secure the first locking mechanism 42. The tensioning/crimping tool may include any suitable tool capable of tensioning and/or securing the first locking mechanism 42.

Engaging the first locking mechanism 42 includes passing an end of the tensile connector 34 through the passage 50 in the crimping device 44, wrapping the tensile connector 34 around the guide device 46, passing the end of the tensile connector 34 back through the passage 50 in the crimping device 44 to thereby form a loop around the guide device 46, and compressing the crimping device 44 over the tensile connector 34 to secure the position of the tensile connector 34 relative to the segmental rod 28 while maintaining the tension in the tensile connector 34. It should be appreciated that a second end of the tensile connector 34 may extend back through the central bores 32 of the segmental members 22 to the opposite axial end of the segmental rod 28, wherein the second end of the tensile connector 34 may be attached to the axial end of the segmental rod 28. As such, both ends of the tensile connector 34 may be fixedly attached to the same axial end of the segmental rod 28, and form an elongated loop that passes through the central bores 32 of the segmental members 22, with the first locking mechanism 42 engaging the tensile connector 34 at an opposite axial end of the segmental rod 28.

If multiple tension zones 61 in the segmental rod 28 are desired, then the method may further include positioning one or more lockable segmental members 62 between the first axial end 36 and the second axial end 38 of the segmental rod 28. The lockable segmental members 62 define at least a first tension zone 61 between the first axial end 36 of the segmental rod 28 and the lockable segmental member 62 and a second tension zone 61 between the lockable segmental member 62 and the second axial end 38 of the segmental rod 28. The method may further include positioning a zone tensioning segmental member 66 within one of the tension zones 61. As described above, the zone tensioning segmental member 66 allows access to the tensile connector 34 within the tension zone 61 so that a second locking mechanism 68 may be engaged to tension the tensile connector 34 within that specific tension zone 61.

The method further includes securing the tensile connector 34 relative to the lockable segmental member 62. The tensile connector 34 may be secured relative to the lockable segmental member 62 in any suitable manner, including but not limited to tightening a set screw onto the tensile connector 34 to secure the tensile connector 34 to the lockable segmental member 62.

Once the tensile connector 34 is secured relative to the lockable segmental member 62, thereby defining the different tension zones 61 on either side thereof, the method further includes tensioning the tensile connector 34 within that tension zone 61 to create a tension in the tensile connector 34 in that tension zone 61 that is different from a tension in the tensile connector 34 in the other tension zones 61. As described above, the tensile connector 34 is tensioned within each tension zone 61 to the desired level to achieve the desired stiffness in the segmental rod 28.

Once the tensile connector 34 is tensioned, the method further includes securing the tensile connector 34 relative to the zone tensioning segmental member 66 with the second locking mechanism 68 to maintain the tension in the tensile connector 34 within that tension zone 61. The second locking mechanism 68 is engaged as described above and similar to the first locking mechanism 42.

While the best modes for carrying out the invention have been described in detail, those familiar with the art to which this invention relates will recognize various alternative designs and embodiments for practicing the invention within the scope of the appended claims.

1. A segmental spinal fixation system comprising:
at least a first segmental member and a second segmental member each including a convex end and a concave end, and each defining a central bore extending longitudinally therethrough, wherein the first segmental member and the second segmental member are disposed adjacent each other to define a segmental rod extending along a longitudinal axis and aligned such that the convex end of one of the first segmental member and the second segmental member is disposed within the concave end of the other of the first segmental member and the second segmental member;
a tensile connector extending through the central bore of each of the first segmental member and the second segmental member;
a locking mechanism disposed at a first axial end of the segmental rod, wherein the locking mechanism includes:
a crimping device defining a passage and disposed adjacent the axial end of the segmental rod; and
a guide device disposed adjacent the crimping device;
wherein the tensile connector extends through the passage of the crimping device, around an outer periphery of the guide device, and extends back through the passage of the crimping device thereby forming a loop around the guide device, with the crimping device disposed in frictional engagement with the tensile connector to secure the tensile connector relative to the segmental rod.

2. A segmental spinal fixation system as set forth in claim 1 wherein the tensile connector is fixedly attached to a second axial end of the segmental rod.

3. A segmental spinal fixation system as set forth in claim 1 wherein the tensile connector includes one of a cable or a wire.

4. A segmental spinal fixation system as set forth in claim 1 wherein each of the first segmental member and the second segmental member include a cylindrical body, with the central bore concentric with a central axis of the cylindrical body.

5. A segmental spinal fixation system as set forth in claim 1 wherein the tensile connector includes a tension force sufficient in magnitude to draw the first segmental member and the second segmental member together and generate a friction force between the first segmental member and the second segmental member sufficient in magnitude to generate a pre-determined stiffness against bending in the segmental rod.

6. A segmental spinal fixation system as set forth in claim 1 further including a plurality of segmental members, wherein the plurality of segmental members includes the first segmental member and the second segmental member, and wherein one of the plurality of segmental members includes a zone tensioning segmental member defining a lateral bore extending laterally from the central bore through a body of the zone tensioning segmental member.

7. A segmental spinal fixation system as set forth in claim 6 wherein the plurality of segmental members includes a lockable segmental member including a fixing device for securing the tensile connector relative to the lockable segmental member, wherein the lockable segmental member is disposed between the first axial end of the segmental rod and a second axial end of the segmental rod to define a plurality of tension zones.

8. A segmental spinal fixation system as set forth in claim 7 wherein the fixing device includes a set screw in threaded engagement with the body of the lockable segmental member and configured for compressing the tensile connector within the central bore to secure the tensile connector in place.

9. A segmental spinal fixation system as set forth in claim 7 further comprising a second locking mechanism disposed adjacent the lateral bore of the zone tensioning segmental member, wherein the tensile connector extends through the central bore of the zone tensioning segmental member, through the lateral bore of the segmental member, through the passage of the crimping device of the second locking mechanism, around an outer periphery of the guide device of the second locking mechanism, back through the passage of the crimping device of the second locking mechanism, back through the lateral bore of the zone tensioning device, and continuing through the central bore of the zone tensioning segmental member, thereby forming a loop around the guide device of the second locking mechanism, with the crimping device of the second locking mechanism disposed in frictional engagement with the tensile connector to secure the tensile connector relative to the zone tensioning segmental member and define a tension in one of the plurality of tension zones.

10. A segmental spinal fixation system as set forth in claim 1 wherein the crimping device is integrally formed with one of the first segmental member and the second segmental member.

11. A segmental spinal fixation system as set forth in claim 10 wherein the crimping device includes a tool engaging mechanism configured for engaging a tensioning/crimping tool.

12. A segmental spinal fixation system as set forth in claim 11 wherein the tool engaging mechanism includes a plurality of detents disposed in a body of the crimping device.

13. A segmental spinal fixation system as set forth in claim 1 wherein the passage of the crimping device includes a first passage for engaging a first portion of the tensile connector and a second passage for engaging a second portion of the tensile connector.

14. A segmental spinal fixation system as set forth in claim 1 wherein the guide device includes a body having an outer surface defining a groove configured for receiving and positioning the tensile connector.

15. A segmental spinal fixation system as set forth in claim 14 wherein the guide device includes a tool engaging mechanism configured for engaging a tensioning/crimping tool.

16. A segmental spinal fixation system as set forth in claim 15 wherein the tool engagement mechanism includes a threaded aperture.

17. A segmental spinal fixation system as set forth in claim 16 wherein the threaded aperture is oriented perpendicularly relative to the longitudinal axis of the segmental rod.

18. A segmental spinal fixation system as set forth in claim 1 wherein each of the concave end and the convex end of the segmental members includes a textured surface configured for engaging each other in frictional engagement to limit movement between the segmental members.

19. A method of positioning a plurality of spinal segments, the method comprising:
attaching a tensile connector to a first axial end of a segmental rod having a plurality of segmental members;
passing the tensile connector through a central bore in each of the plurality of segmental members to position the plurality of segmental members relative to each other and define the segmental rod;
fixing the segmental rod to each of the plurality of spinal segments;
aligning the plurality of spinal segments into a desired position;
tensioning the tensile connector to a tension force sufficient in magnitude to generate a pre-determined stiffness in the segmental rod;
engaging a locking mechanism to secure the tensile connector relative to a second axial end of the segmental rod and to maintain the tension within the tensile connector, wherein engaging the locking mechanism includes;
passing an end of the tensile connector through a crimping device;
wrapping the tensile connector around a guide device;
passing the end of the tensile connector back through the passage in the crimping device to thereby form a loop around the guide device;
compressing the crimping device over the tensile connector to secure the position of the tensile connector relative to the segmental rod while maintaining the tension in the tensile connector.

20. A method as set forth in claim 18 further comprising positioning a lockable segmental member between the first axial end and the second axial end of the segmental rod to define a first tension zone between the first axial end of the segmental rod and the lockable segmental member and a second tension zone between the lockable segmental member and the second axial end of the segmental rod.

21. A method as set forth in claim 19 further comprising positioning a zone tensioning segmental member defining a lateral bore extending from the central bore of the zone tensioning segmental member within the first tension zone.

22. A method as set forth in claim 20 further comprising securing the tensile connector relative to the lockable segmental member.

23. A method as set forth in claim 21 further comprising tensioning the tensile connector within the first tension zone to create a tension in the tensile connector in the first tension zone that is different from a tension in the tensile connector in the second tension zone.

24. A method as set forth in claim 22 further comprising securing the tensile connector relative to the zone tensioning segmental member with a second locking mechanism to maintain the tension in the tensile connector within the first tension zone.

25. A method as set forth in claim 19 wherein tensioning the tensile connector includes pre-tensioning the tensile connector prior to attaching the segmental rod to the spinal segments.

26. A method as set forth in claim 19 wherein the tensile connector includes a first tensile connector and a second tensile connector, and wherein attaching the tensile connector to a first axial end of a segmental rod having a plurality of segmental members includes attaching the first tensile connector and the second tensile connector to the first axial end of the segmental rod along a common plane and separated by 180°.

27. A method as set forth in claim 26 further comprising tensioning one of the first tensile connector or the second tensile connector to steer the first axial end of the segmental rod in one of a first direction or a second direction respectively.

28. A method as set forth in claim 27 wherein tensioning one of the first tensile connector or the second tensile connector is further defined as tensioning one of the first tensile connector or the second tensile connector while the segmental rod is within a human body and prior to fixing the segmental rod to each of the plurality of spinal segments.
